(19) United States
(12) Patent Application Publication
(10) Pub. No.: US 2026/0144484 A1
(43) Pub. Date: May 28, 2026

(54) BODY COMPOSITION PREDICTION BY THE SUMMATION OF REGIONAL ESTIMATES DETERMINED FROM ANATOMICAL SUBREGION GEOMETRY

(71) Applicant: The Government of the United States, as Represented by the Secretary of the Army, Frederick, MD (US)

(72) Inventors: David Patrick Looney, Framingham, MA (US); Adam Whitfield Potter, Walpole, MA (US); Karl Edward Friedl, Frederick, MD (US)

(73) Assignee: The Government of the United States, as Represented by the Secretary of the Army, Frederick, MD (US)

(21) Appl. No.: 19/121,238

(22) PCT Filed: Mar. 11, 2024
(86) PCT No.: PCT/US2024/019392

§ 371 Date: Apr. 15, 2025

Related U.S. Application Data
(60) Provisional application No. 63/453,582, filed on Mar. 21, 2023.

(51) Int. Cl. A61B 5/00; A61B 5/107; G06T 7/00; G06T 7/60

(52) U.S. Cl. CPC A61B 5/4869; A61B 5/0064; A61B 5/1077; A61B 5/7278; G06T 7/0012; G06T 7/60; A61B 2576/00; G06T 2207/30008; G06T 2207/30196

(57) ABSTRACT

A method, system and apparatus determines an individual body composition by the summation of regional estimates determined from anatomical subregion geometry (e.g., lengths, surface areas, volumes). The anatomical subregion geometry may be based on a surface mesh presented by a 3D whole-body scanner. Regional composition estimates are produced by processing subregion geometry combinations by fitted weight values. The regional composition estimates are then summed to produce an individual body composition.

### RELATED APPLICATION

This application claims the benefit of provisional application Ser. No. 63/453,582 filed Mar. 21, 2023 and titled “Body Composition Prediction by the Summation of Regional Estimates Determined from Anatomical Subregion Geometry,” the entire content of which is hereby incorporated by reference.

### STATEMENT OF GOVERNMENT INTEREST

The invention described herein may be manufactured, used and licensed by or for the United States Government.

### BACKGROUND

The present disclosure relates to the prediction of body composition. More particularly, the present disclosure relates to the prediction of body composition using 3-dimensional (3D) scanning systems.

Existing body composition measurement and estimation approaches include air displacement plethysmography, dual-energy x-ray absorptiometry (DXA), hydrodensitometry, and the use of skin calipers and tape measures. These approaches suffer from high costs, long measurement durations and, in the case of DXA, the use of low levels of ionizing radiation.

3D scan-based methods for estimating body composition (e.g., the portions of a body comprising fat and lean, or fat, lean, and bone mineral content (BMC)) generally have been inaccurate, due to methodological limitations. For example, methods based on whole body density assessment generally are imprecise since they rely on principles designed for other instruments and do not account for regional compositional differences. Alternatively, predictive equations can only input a few 3D body measurements due to the limitations of traditional statistical approaches. Consequently, these approaches fail to account for inter-individual variability of body parts that are not included as inputs.

### BRIEF DESCRIPTION OF THE DRAWINGS

FIG. 1 depicts a diagram of steps involved in a 3D surface mesh-based method for calculating individual body composition, in accordance with an embodiment of the present disclosure.

FIG. 2 depicts a division of a 3D surface mesh into body regions, in accordance with an embodiment of the present disclosure.

FIG. 3 depicts a division of a trunk region into subregions, in accordance with an embodiment of the present disclosure.

FIG. 4 depicts an example of the division of a whole-body mesh into regions and subregions, in accordance with an embodiment of the present disclosure.

FIG. 5 depicts a flow chart of an iterative process to determine a best fitting combination of subregion groups for estimating a mass of each body compartment (BC) for each region (arm, head & neck, leg, trunk), in accordance with an embodiment of the present disclosure.

FIG. 6 depicts a block diagram of an apparatus to determine a best fitting combination of subregion groups for estimating a mass of each body compartment (BC) for each region (arm, head & neck, leg, trunk) via an iterative process, in accordance with an embodiment of the present disclosure.

FIG. 7 illustrates an example controller and/or computing environment in which aspects of some of the embodiments of the present disclosure may be implemented, in accordance with embodiments of the present disclosure.

FIG. 8 depicts a block diagram of an apparatus to produce an individual body composition, in accordance with an embodiment of the present disclosure.

### DETAILED DESCRIPTION

Embodiments of the present disclosure will now be described with reference to the drawing figures, in which like reference numerals refer to like parts throughout.

Body composition is known to vary with specialized performance capabilities. Long distance runners adept at running marathons, for example, have a quite different body type and composition than a swimmer, a sumo wrestler, a power lifter, etc. Yet each type of athlete may be considered “fit” for his chosen sport. Body composition as related to specialized performance is an important indicator of physical readiness for a chosen sport or for a specific specialized service.

Certain types of body composition measurement techniques, such as those provided by weight/height tables used to determine an individual's body mass index (BMI); a “tape test” measuring height, weight, circumferential tape measures or like anthropometric measurements, are not particularly relevant and provide only crude indications of physical readiness and fitness.

Embodiments of the present disclosure advantageously provide a description of body shape (physique) and regional body composition, replacing the total body composition typically produced by other methods and representing an improvement over an existing technological process. For example, an organization may use, at present, an assessment of body composition that focuses on modifiable sites of fat deposition such as abdominal and hip fat instead of including other sites that have a stronger genetic determinant such as centripetal (i.e., upper back and arms) sites, or where fat is mobilized only in specific conditions such as pregnancy/post-partum (thigh fat). Embodiments of the present disclosure advantageously evaluate muscle mass, supporting, e.g., the use of a minimum lean mass standard that would promote safe physical training. Similarly, from a general health perspective, embodiments of the present disclosure advantageously enable superior quantifiable assessments in body composition that allow for improved targeted assessments of human health compared to traditional methods (e.g., height, weight, or crude indices such as body mass index (BMI)). Further, the improved assessments of health provided by embodiments of the present disclosure may be advantageously applied during the treatment of obesity, anorexia, and other medical conditions.

Additionally, some professional sports teams are already assessing regional muscle mass of some of their contracted players as a condition of continued play, but currently must conduct this assessment using expensive DXA techniques that also involve exposure of the player to low levels of ionizing radiation. Embodiments of the present disclosure, using the superior three-compartment model (i.e., fat, lean and BMC) of DXA, advantageously are more accurate than density-based methods that must interpret two-compartment models (i.e., fat and lean, and miss variance introduced by bone), and also avoid exposure of the subject to the low levels of ionizing radiation of DXA.

Advantageously, embodiments of the present disclosure do not require circumference or mass measurements and are capable of accurate regional and whole-body estimates of BMC, fat, and lean tissue.

In one embodiment, a system includes a 3D scanner, a memory and a processor configured to calculate an individual body composition by the summation of regional estimates determined from anatomical subregion geometry. The anatomical subregion geometry may be based on a surface mesh presented by the 3D scanner. The 3D scanner is a body surface scanner capable of generating a three-dimensional (3D), whole-body mesh (e.g., an SS20 3D body scanner from Size Stream LLC, Cory, NC). Based at least in part on the surface mesh presented by the 3D scanner, the processor identifies anatomical subregion geometry (e.g., lengths, surface areas, volumes), produces regional estimates by processing, such as by multiplying, the subregion geometries by weights stored in the memory or other storage, and then sums the regional estimates to produce an individual body composition.

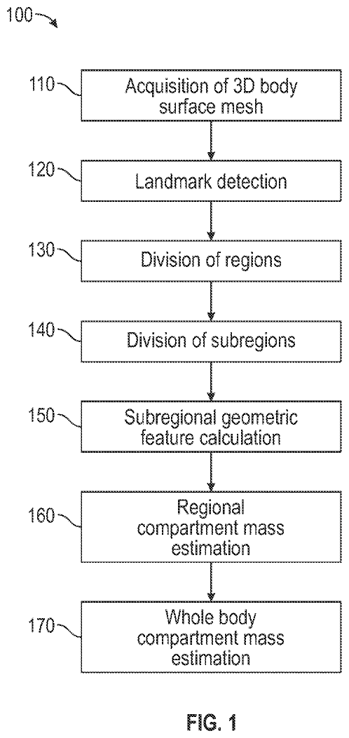

FIG. 1 depicts flow diagram 100 of a calculation of an individual body composition, in accordance with an embodiment of the present disclosure. At 110, a 3D whole-body surface mesh is acquired. The 3D whole-body surface mesh may be acquired from a body surface scanner, e.g., an SS20 3D body scanner as described supra, or by other means, e.g., a human body surface mesh generated by computer.

In an embodiment, the mesh is normalized by median-centering the x (width) and z (depth) coordinates before scaling the x, y (height), and z coordinates relative to the maximum y coordinate.

At 120, body landmarks are detected. Landmarks can be input from existing scanner software or identified by applying landmark-specific search functions within defined normalized x, y, and z limits. These limits are initially set to values that cover beyond the range of landmark coordinates in the combined dataset up to anatomical extremes. However, these initial values are progressively updated as adjacent landmarks are identified. Table 1 summarizes landmarks, including brief descriptions, according to an embodiment; other landmarks and combinations of landmarks are also contemplated.

TABLE 1
Landmarks and Descriptions.

| Landmark | Description |
|---|---|
| Abdomen | Midpoint of trunk between seat and waist. |
| Ankle (Left & Right) | Midpoint of leg at ankle joint. |
| Base | Centered maximum z coordinate where neck |
| | meets shoulders. |
| Calf (Left & Right) | Midpoint of leg between knee and ankle. |
| Chest | Midpoint of trunk at widest part between waist |
| | and clavicles near sternum. |
| Chest, Lower | Midpoint of trunk between chest and waist. |
| Chest, Upper | Midpoint of trunk between base of neck and |
| | chest. |
| Crotch | Minimum y coordinate of midpoint where trunk |
| | intersects with thighs. |
| Elbow (Left & Right) | Midpoint of elbow joint of arm. |
| Forehead | Midpoint of head at widest part of skull above |
| | eyes. |
| Heel (Left & Right) | Minimum z coordinate at minimum y coordinate |
| | of body. |
| Knee, Anterior | Anterior midpoint of knee joint. |
| Knee, Posterior | Posterior midpoint of leg between knee and calf. |
| Knuckle (Left & Right) | Centered minimum y coordinate of arm. |
| Midneck, Anterior | Midpoint of anterior neck between chin and |
| | base. |
| Midneck, Posterior | Midpoint of posterior neck between chin and |
| | base. |
| Seat | Midpoint of trunk between crotch and abdomen |
| | near iliac crest. |
| Shoulder (Left & Right) | Midpoint of shoulder joint where upper arm |
| | intersects chest. |
| Thigh (Left & Right) | Midpoint of leg between crotch and knee. |
| Thigh, Upper (Left & Right) | Midpoint of leg between crotch and thigh. |
| Top | Maximum y coordinate of body. |
| Waist | Midpoint of trunk between seat and bust. |
| Wrist (Left & Right) | Midpoint of arm at wrist joint. |

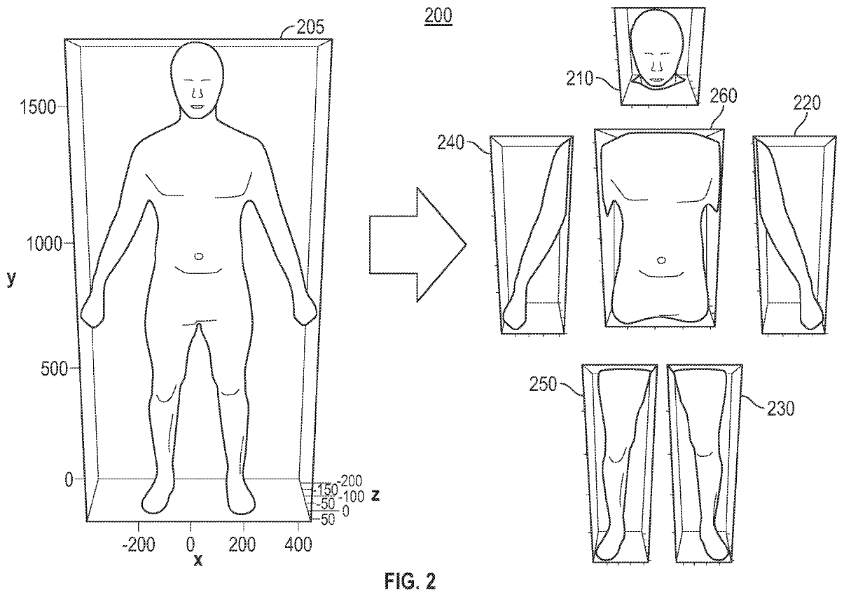

At 130, the acquired 3D body mesh is divided into at least six regions: head and neck; left arm; left leg; right arm; right leg; and trunk, as depicted in FIG. 2. FIG. 2 depicts a division of a 3D surface mesh into body regions, in accordance with an embodiment of the present disclosure.

FIG. 2 depicts a division 200 of acquired 3D body mesh 205 into six regions, according to an embodiment of the present disclosure. Acquired 3D body mesh 205 is divided into head and neck region 210, left arm region 220, left leg region 230, right arm region 240, right leg region 250, and trunk region 260.

Returning to FIG. 1, at 140 each region is further divided into subregions based on key anatomical landmarks and optimized predictive relationships with body compartments. The exact number of subregions is specific to each body compartment within each region.

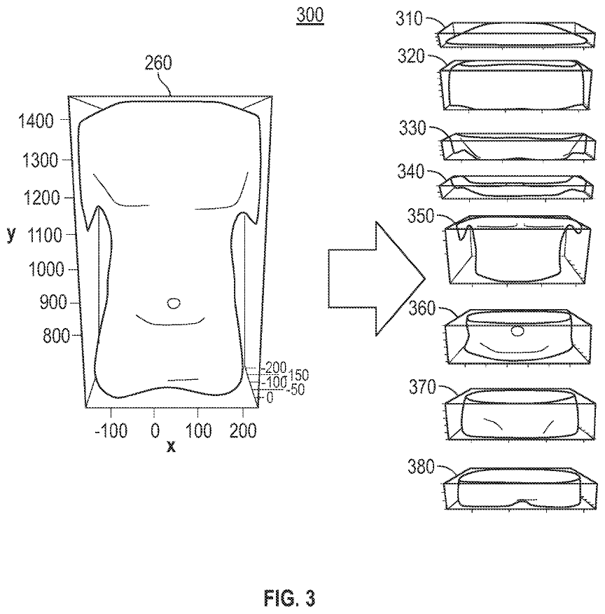

FIG. 3 depicts an example subdivision 300 of trunk region 260 into eight subregions, in accordance with an embodiment of the present disclosure. Trunk region 260 is subdivided into, e.g., eight subregions: subregion T1 310, subregion T2 320, subregion T3 330, subregion T4 340, subregion T5 350, subregion T6 360, subregion T7 370, and subregion T8 380. Example subregions and dividing landmarks for arm and head & neck regions (Table 2) as well as leg and trunk regions (Table 3) are shown below.

TABLE 2
Example subregions and dividing landmarks
for arm, and head & neck regions.

| Region | Subregion | Dividing Landmark |
|---|---|---|
| Arm (A) | A₁ | Knuckle |
| | A₂ | Wrist |
| | A₃ | Elbow |
| | | Shoulder |
| Head & Neck (H) | H₁ | Midneck Anterior |
| | H₂ | Midneck Posterior |
| | H₃ | Forehead |
| | | Top |

TABLE 3
Example subregions and dividing landmarks
for leg and trunk regions.

| Region | Subregion | Dividing Landmark |
|---|---|---|
| Leg (L) | L₁ | Heel |
| | L₂ | Ankle |
| | L₃ | Calf |
| | L₄ | Knee, Posterior |
| | L₅ | Knee, Anterior |
| | L₆ | Thigh |
| | L₇ | Thigh, Upper |
| | | Crotch |
| Trunk (T) | T₁ | Crotch |
| | T₂ | Seat |
| | T₃ | Abdomen |
| | T₄ | Waist |
| | T₅ | Chest, Lower |
| | T₆ | Chest |
| | T₇ | Chest, Upper |
| | T₈ | Base |
| | | Midneck, Anterior |

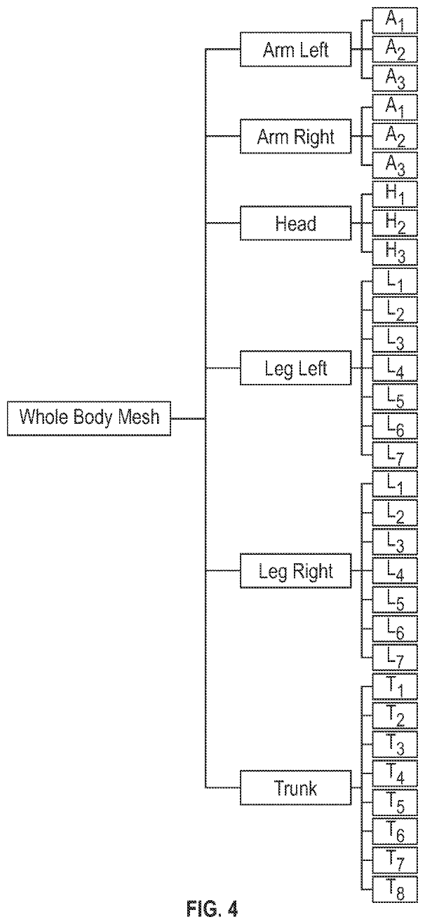

FIG. 4 depicts an example of the division of a whole-body mesh into regions and subregions, in accordance with an embodiment of the present disclosure.

Returning to FIG. 1, at 150 subregional geometric features (e.g., length, surface area, volume, etc.) of each subregion are determined using default functions found in 3D mesh software, open-source 3D mesh analysis packages, etc.

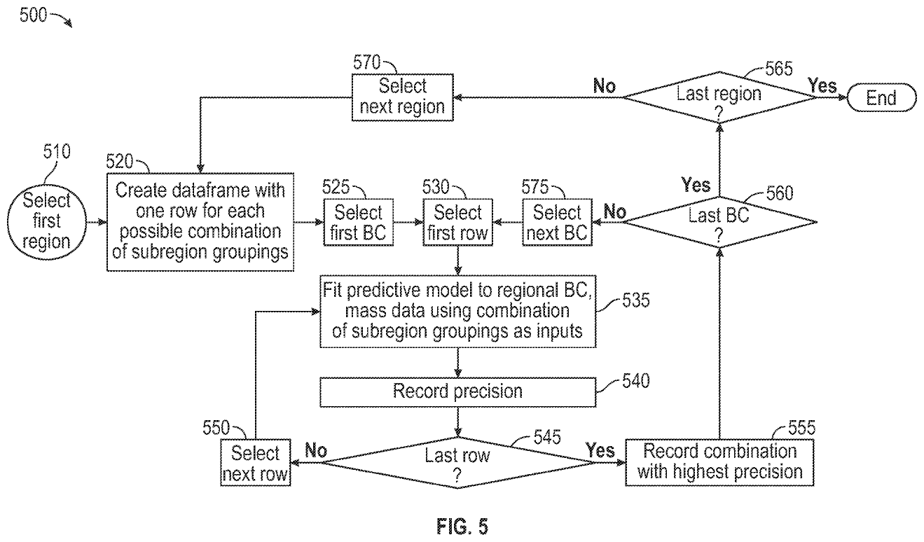

At 160, the calculated geometric features of each subregion and their interactions are used to calculate the regional mass of each body compartment. For each tissue type, features (length, surface area, etc.) of each subregion are multiplied by a pre-fitted coefficient and then summed together to estimate the regional mass. The equation formula for each body compartment (i.e., fat, lean, BMC) within a region are fit on an aggregated database of 3D mesh and DXA data (Table 4) through an iterative process (FIG. 5). As depicted in FIG. 5, the base formula of the iterative process includes each subregion separately; while subsequent iterations test whether combining adjacent subregions improves model accuracy and precision, as quantified by the root mean square deviation (RMSD). In Table 4, one coefficient is fit for each individual subregion or parenthetical group of combined subregions.

TABLE 4
Best fitting formulae determined during initial analysis. ( ), combined subregions;
₁₋₈, subregion number; A, arm region; H, head region; L, leg region; T, trunk region;
d, distance (cm); s, surface area (cm²); v, volume (L).

| Region | Compart | Formula |
|---|---|---|
| Arm | BMC | dA₁ + dA₂ + dA₃ + (sA₁ + sA₂ + sA₃) + (vA₁ + vA₂ + vA₃) |
| (A) | Fat | dA₁ + (dA₂ + dA₃) + (sA₁ + sA₂) + sA₃ + vA₁ + vA₂ + vA₃ |
| | Lean | dA₁ + dA₂ + dA₃ + sA₁ + sA₂ + sA₃ + vA₁ + vA₂ + vA₃ |
| Head | BMC | (dH₁ + dH₂ + dH₃) + (sH₁ + sH₂) + sH₃ + (vH₁ + vH₂) + vH₃ |
| (H) | Fat | (dH₁ + dH₂) + dH₃ + (sH₁ + sH₂) + sH₃ + (vH₁ + vH₂) + vH₃ |
| | Lean | (dH₁ + dH₂ + dH₃) + (sH₁ + sH₂ + sH₃) + (vH₁ + vH₂ + vH₃) |
| Leg | BMC | dL₁ + dL₂ + dL₃ + dL₄ + dL₅ + (dL₆ + dL₇) + (sL₁ + sL₂ + sL₃) + sL₄ + sL₅ + |
| (L) | | sL₆ + sL₇ + (vL₁ + vL₂) + vL₃ + vL₄ + vL₅ + (vL₆ + vL₇) |
| | Fat | dL₁ + (dL₂ + dL₃) + (dL₄ + dL₅ + dL₆ + dL₇) + (sL₁ + sL₂) + sL₃ + sL₄ + (sL₅ + |
| | | sL₆) + sL₇ + vL₁ + vL₂ + vL₃ + (vL₄ + vL₅) + vL₆ + vL₇ |
| | Lean | dL₁ + dL₂ + (dL₃ + dL₄) + (dL₅ + dL₆ + dL₇) + (sL₁ + sL₂) + (sL₃ + sL₄) + sL₅ + |
| | | sL₆ + sL₇ + vL₁ + vL₂ + vL₃ + vL₄ + vL₅ + vL₆ + vL₇ |
| Trunk | BMC | (dT₁ + dT₂ + dT₃) + (dT₄ + dT₈) + (dT₆ + dT₇ + dT₈) + sT₁ + (sT₂ + sT₃ + sT₄) + |
| (T) | | (sT₅ + sT₆) + (sT₇ + sT₈) + vT₁ + (vT₂ + vT₃ + vT₄ + vT₅ + vT₆ + vT₇) + vT₈ |
| | Fat | (dT₁ + dT₂ + dT₃) + dT₄ + dT₅ + dT₆ + (dT₇ + dT₈) + sT₁ + (sT₂ + sT₃ + sT₄) + |
| | | sT₅ + sT₆ + (sT₇ + sT₈) + vT₁ + (vT₂ + vT₃ + vT₄) + vT₅ + (vT₆ + vT₇) + vT₈ |
| | Lean | (dT₁ + dT₂ + dT₃) + (dT₄ + dT₅) + dT₆ + (dT₇ + dT₈) + sT₁ + (sT₂ + sT₃ + sT₄) + |
| | | sT₅ + sT₆ + (sT₇ + sT₈) + vT₁ + (vT₂ + vT₃ + vT₄) + vT₅ + (vT₆ + vT₇ + vT₈) |

An iterative process to determine a best-fit combination of subregion groups for estimating the mass of each body compartment (BC) for each region (arm, head & neck, leg, trunk), in accordance with an embodiment, is depicted in FIG. 5. A first region is selected at 510. For that first region, a dataframe is created at 520, having one row for each possible combination of subregion groupings. For example, the Arm region has three subregions; the distance parameter may be dA₁+dA₂+dA₃, dA₁+(dA₂+dA₃), (dA₁+dA₂)+dA₃, (dA₁+dA₃)+dA₂, or (dA₁+dA₂+dA₃), using 3, 2, 2, 2, or 1 fitting weights, respectively, and there are similar (but independent) combinations for the surface area and volume parameters. Next, a first BC is selected at 525, then a first row of the dataframe for that BC at 530. A predictive model is then fit to the regional BC mass data, using the combination of subregion groupings as inputs at 535. The resulting accuracy is then recorded at 540, such as by storing it in memory or storage. If the present row is not the last row of the present BC as determined at 545, the next row is selected at 550 and flow returns to the predictive model fit at 535. However, if the present row is the last row of the present BC, the combination of the subregion groupings having the highest accuracy is recorded in memory or storage at 555. If the present BC is not the last BC as determined at 560, the next BC is selected at 575, the first row of that BC is selected at 530, and flow returns to the predictive model fit at 535. However, if the present row is the last BC, the present region is evaluated at 565. If the present region is not the last region, the next region is selected at 570 and a new dataframe is created at 520. However, if the present region is the last region as determined at 565, the process is complete.

Tables 5-8 show example model coefficients for the arm, head & neck, leg, and trunk regions for estimating mass of each body compartment that were fit during a preliminary analysis.

TABLE 5
Example subregion coefficients for estimating mass of each body
compartment within left and right arm regions. Coefficients
spanning across multiple subregions/rows are fit to the sum of the
subregions within that span. ₁₋₃, subregion number; A, arm region;
BMC, bone mineral content; d, distance (cm); Intercept, model intercept;
s, surface area (cm²); v, volume (L).

| Subregion | BMC | Fat | Lean |
|---|---|---|---|
| Intercept | 0.003643 | 3.245876 | 0.23553 |
| dA₁ | −0.000292 | −0.013438 | −0.00067 |
| dA₂ | 0.000105 | −0.007369 | 0.012264 |
| dA₃ | −0.000339 | | 0.001645 |
| sA₁ | 0.000181 | 0.00431 | 0.001568 |
| sA₂ | | | −0.013676 |
| sA₃ | | 0.000979 | 0.001316 |
| vA₁ | 0.000003 | −0.000469 | 0.000137 |
| vA₂ | | −0.001652 | 0.005517 |
| vA₃ | | 0.000079 | 0.000098 |

TABLE 6
Example subregion coefficients for estimating mass of each body
compartment within head and neck regions. Coefficients spanning
across multiple subregions/rows are fit to the sum of the subregions
within that span. ₁₋₃, subregion number; H, head and neck region; BMC,
bone mineral content; d, distance (cm); Intercept, model intercept;
s, surface area (cm²); v, volume (L).

| Subregion | BMC | Fat | Lean |
|---|---|---|---|
| Intercept | 0.272878 | 1.08651 | 1.031465 |
| dH₁ | 0.00108 | −0.001631 | 0.00143 |
| dH₂ | | | |
| dH₃ | | 0.003236 | |
| sH₁ | −0.000383 | −0.000808 | −0.002049 |
| sH₂ | | | |
| sH₃ | −0.003236 | −0.004941 | |
| vH₁ | 0.00016 | 0.000366 | 0.001003 |
| vH₂ | | | |
| vH₃ | 0.000993 | 0.001294 | |

TABLE 7
Example subregion coefficients for estimating mass of each body
compartment within head and neck regions. Coefficients spanning
across multiple subregions/rows are fit to the sum of the subregions
within that span. ₁₋₇, subregion number; L, leg region; BMC, bone
mineral content; d, distance (cm); Intercept, model intercept; s,
surface area (cm²); v, volume (L).

| Subregion | BMC | Fat | Lean |
|---|---|---|---|
| Intercept | 1.556479 | −14.135775 | 20.808568 |
| dL₁ | 0.003805 | 0.011738 | 0.012856 |
| dL₂ | 0.001794 | −0.0026 | 0.031481 |
| dL₃ | −0.000905 | | −0.010531 |
| dL₄ | 0.018134 | 0.056679 | |
| dL₅ | −0.000672 | | −0.092196 |
| dL₆ | −0.015082 | | |
| dL₇ | | | |
| sL₁ | −0.000215 | −0.000892 | −0.006669 |
| sL₂ | | | |
| sL₃ | | 0.019529 | −0.031163 |
| sL₄ | −0.010393 | −0.03608 | |
| sL₅ | 0.003845 | −0.014687 | 0.04969 |
| sL₆ | 0.001009 | | 0.006381 |
| sL₇ | −0.002948 | 0.025146 | −0.013998 |
| vL₁ | −0.000013 | −0.000616 | 0.002748 |
| vL₂ | | 0.001256 | −0.00088 |
| vL₃ | 0.000246 | −0.006598 | 0.012213 |
| vL₄ | 0.002004 | 0.006298 | 0.011025 |
| vL₅ | −0.000434 | | −0.007403 |
| vL₆ | 0.000021 | −0.002048 | 0.002306 |
| vL₇ | | 0.003581 | −0.002642 |

TABLE 8
Example subregion coefficients for estimating mass of each body
compartment within head and neck regions. Coefficients spanning
across multiple subregions/rows are fit to the sum of the subregions
within that span. ₁₋₈, subregion number; T, trunk region; BMC, bone
mineral content; d, distance (cm); Intercept, model intercept; s,
surface area (cm²); v, volume (L).

| Subregion | BMC | Fat | Lean |
|---|---|---|---|
| Intercept | −1.483027 | 7.909679 | −22.926939 |
| dT₁ | 0.021496 | −0.242147 | 0.456113 |
| dT₂ | | | |
| dT₃ | | | |
| dT₄ | −0.01958 | 0.213256 | −0.492304 |
| dT₅ | | 0.319582 | |
| dT₆ | 0.000373 | 0.015409 | −0.007763 |
| dT₇ | | −0.046723 | 0.07848 |
| dT₈ | | | |
| sT₁ | −0.00263 | 0.025295 | −0.044694 |
| sT₂ | 0.000451 | −0.004414 | 0.013087 |
| sT₃ | | | |
| sT₄ | | | |
| sT₅ | 0.000171 | −0.019688 | 0.022672 |
| sT₆ | | −0.002236 | 0.005616 |
| sT₇ | −0.000121 | 0.004913 | −0.006316 |
| sT₈ | | | |
| vT₁ | 0.000187 | −0.000539 | 0.001725 |
| vT₂ | −0.000016 | 0.001096 | −0.000602 |
| vT₃ | | | |
| vT₄ | | | |
| vT₅ | | 0.002983 | −0.003623 |
| vT₆ | | −0.000939 | 0.001742 |
| vT₇ | | | |
| vT₈ | 0.000245 | 0.001363 | |

Returning to FIG. 1, at 170 estimates of each whole body compartment mass are made. The total mass of each body compartment is the sum of its regional estimates. Whole body mass of the BMC (mBMC), Fat (mFat), and Lean (mLean) body compartments is calculated using the following equations.

$$mBMC ( kg ) = mBMCArmLeft + mBMCArmRight + mBMCHead + mBMCLegLeft + mBMCLegRight + mBMCTrunk \qquad ( 1 )$$

$$mFat ( kg ) = mFatArmLeft + mFatArmRight + mFatHead + mFatLegLeft + mFatLegRight + mFatTrunk \qquad ( 2 )$$

$$mLean ( kg ) = mLeanArmLeft + mLeanArmRight + mLeanHead + mLeanLegLeft + mLeanLegRight + mLeanTrunk \qquad ( 3 )$$

Body composition is calculated by dividing the mass of each body compartment by the total mass of all body compartments. The percentage of body mass for the BMC (pBMC), Fat (pFat), and Lean (pLean) compartments is calculated using the following equations.

$$pBMC ( \% body mass ) = 100 * mBMC / ( mBMC + mFat + mLean ) \qquad ( 4 )$$

$$pFat ( \% body mass ) = 100 * mFat / ( mBMC + mFat + mLean ) \qquad ( 5 )$$

$$pLean ( \% body mass ) = 100 * mLean / ( mBMC + mFat + mLean ) \qquad ( 6 )$$

A preliminary analysis was conducted on data collected from twenty-nine healthy individual males. Table 9 displays the preliminary results on the accuracy of total body composition estimates, in accordance with an embodiment of the present disclosure, against the gold standard DXA. Table 10 displays the preliminary results on the accuracy of regional and total body mass estimates for the three body compartments (BMC, fat, and lean mass). The same iterative development strategy may be applied to female subjects, or indeed subjects drawn from any population.

As shown in Table 9, and in accordance with an embodiment of the present disclosure, the disclosed method has a body fat compartment root-mean-square deviation (RMSD) of 1.42% body mass—over 2.5 times less than the existing Size Stream 3D scanner body fat prediction algorithm (3.95% body mass).

TABLE 9
Results from preliminary analysis on the accuracy of estimating
each body compartment as a percentage of body mass (%)
against the gold standard dual-energy x-ray absorptiometry
(DXA). BMC, bone mineral content; CCC, concordance correlation
coefficient; RMSD, root-mean-square deviation.

| | RMSD | |
|---|---|---|
| Compartment | (%) | CCC |
| BMC | 0.18 | 0.82 |
| Fat | 1.42 | 0.96 |
| Lean | 1.32 | 0.96 |

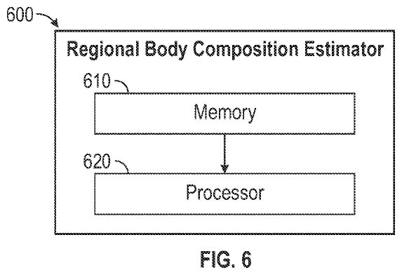

FIG. 6 depicts a block diagram of aa regional body composition estimator apparatus 600 used to determine a best fitting combination of subregion groups for estimating a mass of each body compartment (BC) for each region (arms, head & neck, legs, trunk) via an iterative process, in accordance with an embodiment of the present disclosure.

The regional body composition estimator apparatus 600 includes a processor 620, coupled to a memory 610, i.e. storage. The processor 620 of the regional body composition estimator is configured to receive one or more anatomical subregion geometries (e.g., distances, surface areas, volumes, etc.) and associated regional body compartment mass values from the memory, fit a predictive model to the regional body compartment mass values using combinations of subregion groupings as inputs via an iterative process, and determine the combination of subregion groupings having a best fit in the memory. The combination of subregion groupings having the best fit, with the associated weights, may be stored in the memory 610.

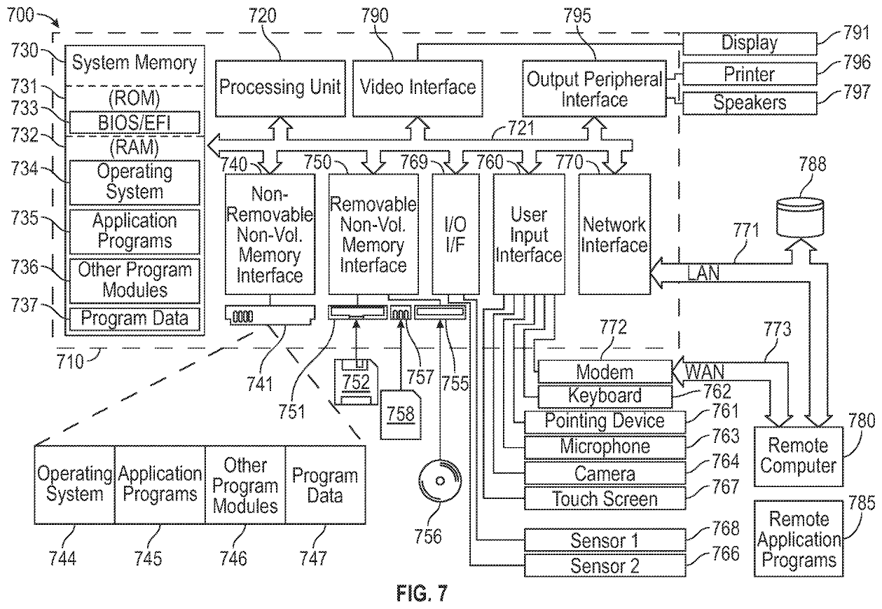

The processor 620 and/or the memory 610 of this regional body composition estimator apparatus 600 may reside within a 3D body scanner, such as described above, that generates the one or more anatomical subregion geometries and associated regional body compartment mass values. Alternately, the processor and/or the memory of the regional body composition estimator may reside within a computing device, described below in connection with FIG. 7. Further, the processor and/or the memory of the regional body composition estimator may reside within the same or different points within a distributed computing environment, described below in connection with FIG. 7. In other words, it is not a requirement of the present disclosure that the processor and memory of a regional body composition estimator apparatus for determining a best fitting combination of subregion groups for estimating a mass of body compartments reside together on the same hardware platform.

FIG. 7 illustrates an example controller and/or computing environment on which aspects of some embodiments, including the processor and memory of a regional body composition estimator, may be implemented. The computing environment 700 is only one example of a computing environment and is not intended to suggest any limitation as to the scope of use or functionality of the claimed subject matter. Neither should the computing environment 700 be interpreted as having any dependency or requirement relating to any one or combination of components illustrated in the example operating environment 700.

Embodiments are operational with numerous other general purpose or special purpose computing system environments or configurations. Examples of well-known computing systems, environments, and/or configurations that may be suitable for use with various embodiments include, but are not limited to, embedded computing systems, personal computers, server computers, mobile devices, hand-held or laptop devices, multiprocessor systems, microprocessor-based systems, set top boxes, programmable consumer electronics, medical device, network PCs, minicomputers, mainframe computers, cloud services, telephonic systems, distributed computing environments that include any of the above systems or devices, and the like.

Embodiments may be described in the general context of computer executable instructions, such as program modules, being executed by computing capable devices. Generally, program modules include routines, programs, objects, components, data structures, etc. that perform particular tasks or implement particular abstract data types. Some embodiments may be designed to be practiced in distributed computing environments where tasks are performed by remote processing devices that are linked through a communications network. In a distributed computing environment, program modules may be located in both local and remote computer storage media including memory storage devices.

With reference to FIG. 7, an example system for implementing some embodiments includes a computing device 710. Components of computing device 710 may include, but are not limited to, a processing unit 720, a system memory 730, and a system bus 721 that couples various system components including the system memory to the processing unit 720. It is understood and contemplated that any of system memory 730 may be used to store weights used by the processor of a regional body composition estimator system in order to generate anatomical subregion geometries based, at least in part, on a surface mesh received from a 3D body scanner.

Computing device 710 may comprise a variety of computer readable media. Computer readable media may be any available media that can be accessed by computing device 710 and includes both volatile and nonvolatile media, and removable and non-removable media. It is understood and contemplated that any computer readable media used by a computing device 710 may store weights used by the processor element of a regional body composition estimator, whether or not resident on computing device 710, to generate anatomical subregion geometries based, at least in part, on a surface mesh received from a 3D body scanner.

By way of example, and not limitation, computer readable media may comprise computer storage media and communication media. Computer storage media may comprise volatile and/or nonvolatile, and/or removable and/or non-removable media implemented in any method or technology for storage of information such as computer readable instructions, data structures, program modules or other data. Computer storage media comprises, but is not limited to, random access memory (RAM), read-only memory (ROM), electrically erasable programmable read-only memory (EEPROM), flash memory or other memory technology, compact disc read-only memory (CD-ROM), digital versatile disks (DVD) or other optical disk storage, magnetic cassettes, magnetic tape, magnetic disk storage or other magnetic storage devices, or any other medium which can be used to store the desired information and which can be accessed by computing device 710. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared and other wireless media configured to communicate modulated data signal(s). Combinations of any of the above should also be included within the scope of computer readable media.

System memory 730 includes computer storage media in the form of volatile and/or nonvolatile memory such as ROM 731 and RAM 732. A basic input/output system 733 (BIOS), containing the basic routines that help to transfer information between elements within computing device 710, such as during start-up, is typically stored in ROM 731. RAM 732 typically contains data and/or program modules that are immediately accessible to and/or presently being operated on by processing unit 720. By way of example, and not limitation, FIG. 7 illustrates operating system 734, application programs 735, other program modules 736, and program data 737 that may be stored in RAM 732.

Computing device 710 may also include other removable/non-removable volatile/nonvolatile computer storage media, any of which may be used to store weights used by the processor of a regional body composition estimator system in order to generate anatomical subregion geometries based, at least in part, on a surface mesh received from a 3D body scanner. By way of example only, FIG. 7 illustrates a hard disk drive 741 that reads from or writes to non-removable, nonvolatile magnetic media, a magnetic disk drive 751 that reads from or writes to a removable, nonvolatile magnetic disk 752, a flash drive reader 757 that reads flash drive 758, and an optical disk drive 755 that reads from or writes to a removable, nonvolatile optical disk 756 such as a Compact Disc Read Only Memory (CD ROM), Digital Versatile Disc (DVD), Blue-ray Disc™ (BD) or other optical media. Other removable/non-removable, volatile/nonvolatile computer storage media that can be used in the example operating environment include, but are not limited to, magnetic tape cassettes, flash memory cards, digital versatile disks, digital video tape, solid state RAM, solid state ROM, and the like. The hard disk drive 741 is typically connected to the system bus 721 through a non-removable memory interface such as interface 740, and magnetic disk drive 751 and optical disk drive 755 are typically connected to the system bus 721 by a removable memory interface, such as interface 750.

The drives and their associated computer storage media discussed above and illustrated in FIG. 7 provide storage of computer readable instructions, data structures, program modules and other data for computing device 710. In FIG. 7, for example, hard disk drive 741 is illustrated as storing operating system 744, application programs 745, program data 747, and other program modules 746. Additionally, for example, non-volatile memory may include instructions, for example, to discover and configure IT device(s); to create device neutral user interface command(s); combinations thereof, and/or the like.

A user may enter commands and information into computing device 710 through input devices such as a keyboard 762, a microphone 763, a camera 764, touch screen 767, and a pointing device 761, such as a mouse, trackball or touch pad. These and other input devices are often connected to the processing unit 720 through a user input interface 760 that is coupled to the system bus, but may be connected by other interface and bus structures, such as a parallel port, a game port and/or a universal serial bus (USB).

Sensors, such as sensor 1 768 and sensor 2 766, may be connected to the system bus 721 via an Input/Output Interface (I/O I/F) 769. Examples of sensor(s) 766, 768 include a microphone, an accelerometer, an inertial navigation unit, a piezoelectric crystal, and/or the like. Additionally, and in connection with an apparatus and/or system described within the disclosure, Sensors 766, 768 may be part of or include a 3D body scanner that generates a surface mesh of an individual who has been scanned. A monitor 791 or other type of display device may also be connected to the system bus 721 via an interface, such as a video interface 790. Other devices, such as, for example, speakers 797 and printer 796 may be connected to the system via peripheral interface 795.

Computing device 710 may be operated in a networked environment using logical connections to one or more remote computers, such as a remote computer 780. The remote computer 780 may be a personal computer, a mobile device, a hand-held device, a server, a router, a network PC, a medical device, a peer device or other common network node, and typically includes many or all of the elements described above relative to computing device 710. The logical connections depicted in FIG. 7 include a local area network (LAN) 771 and a wide area network (WAN) 773, but may also include other networks such as, for example, a cellular network. Such networking environments are commonplace in offices, enterprise-wide computer networks, intranets and the Internet.

When used in a LAN networking environment, computing device 710 may be connected to the LAN 771 through a network interface or adapter 770. When used in a WAN networking environment, computing device 710 typically includes a modem 772 or other means for establishing communications over the WAN 773, such as the Internet. The modem 772, which may be internal or external, may be connected to the system bus 721 via the user input interface 760, or other appropriate mechanism. The modem 772 may be wired or wireless. Examples of wireless devices may comprise, but are limited to: Wi-Fi, Near-field Communication (NFC) and Bluetooth™. In a networked environment, program modules depicted relative to computing device 710, or portions thereof, may be stored in the remote memory storage device 788. By way of example, and not limitation, FIG. 7 illustrates remote application programs 785 as residing on remote computer 780. It will be appreciated that the network connections shown are exemplary and other means of establishing a communications link between the computers may be used. Additionally, for example, LAN 771 and WAN 773 may provide a network interface to communicate with other distributed infrastructure management device(s); with IT device(s); with users remotely accessing the User Input Interface 760; combinations thereof, and/or the like.

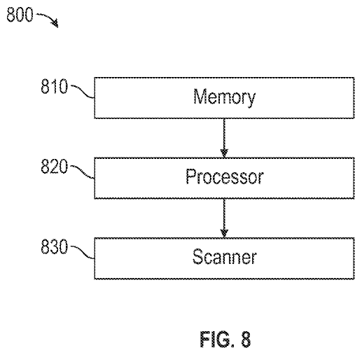

FIG. 8 depicts a block diagram of a regional body composition estimator system 800 to produce an individual body composition, in accordance with an embodiment of the present disclosure.

The regional body composition estimator system 800 includes a 3D body scanner 830, a memory 810, and a processor 820 coupled to the scanner 830 and the memory 810. The processor 820 of the regional body composition estimator system 800 is configured to generate one or more anatomical subregion geometries (e.g., distances, surface areas, volumes, etc.) based, at least in part, on a surface mesh received from the body scanner, produce regional composition estimates by processing, such as multiplying, the subregion geometries by weights received from the memory, and sum the regional composition estimates to produce an individual body composition. The processor 820 may be further configured to produce regional composition estimates by processing, i.e. multiplying, combinations of subregion geometries by weights received from the memory. The various configurations of such a regional body composition estimator system 800, and in particular to the arrangement of the processor, memory and scanner elements of the regional body composition estimator system have been described at length above, with reference to FIG. 7.

The following embodiments are combinable.

Therefore, in one embodiment of the disclosure, an example system is provided to include a three-dimensional (3D) body scanner; and a regional body composition estimator, coupled to the 3D body scanner, having a processor configured to generate one or more anatomical subregion geometries based, at least in part, on a surface mesh received from the 3D body scanner, produce regional composition estimates by processing the subregion geometries by weights, and produce an individual body composition from the regional composition estimates.

In another embodiment of the system, the system where the individual body composition includes an estimate of at least one of fat, lean, and bone mineral content as a portion of total body mass.

In an additional embodiment of the system, the system where processing the subregion geometries includes multiplying subregion geometry combinations.

In another embodiment of the system, the system includes a memory that stores the weights, where the regional body composition estimator processes the subregion geometries by weights received from the memory.

In an additional embodiment of the system, the system the individual body composition is produced by summing the regional composition estimates.

In another embodiment of the disclosure, an example apparatus is provided to include a regional body composition estimator apparatus, comprising regional body composition estimator having: a memory; and a processor, coupled to the memory, configured to: receive one or more anatomical subregion geometries and associated regional body compartment mass values from the memory, fit a predictive model to the regional body compartment mass values using combinations of subregion groupings as inputs, and determine the combination of subregion groupings having a best fit.

In another embodiment of the apparatus, the apparatus includes a processor further configured to store the combination of subregion groupings having the best fit in the memory.

In an additional embodiment of the apparatus, the apparatus includes the memory and the processor of the regional body composition estimator apparatus reside within a body scanner that generates the one or more anatomical subregion geometries and associated regional body compartment mass values.

In another embodiment of the apparatus, the apparatus includes that at least one or more of the memory and the processor of the regional body composition estimator apparatus reside within a computing device or where at least one or more of the memory and the processor reside within a distributed computing environment.

In another embodiment of the disclosure, an example method is provided that includes receiving one or more anatomical subregion geometries and associated regional body compartment mass values; iteratively fitting a predictive model to the regional body compartment mass values using combinations of subregion groupings as inputs; and determining the combination of subregion groupings having a best fit.

In another embodiment of the method, a method includes recording the combination of subregion groupings having the best fit in storage.

In a further embodiment of the method, the method includes generating one or more anatomical subregion geometries based, at least in part, on a surface mesh received from the 3D body scanner.

In another embodiment of the disclosure, an example method is provided to include generating one or more anatomical subregion geometries based, at least in part, on a surface mesh producing regional composition estimates by processing the subregion geometries by weights and producing an individual body composition from the regional composition estimates.

In another embodiment of the method, the method of processing the subregion geometries includes multiplying subregion geometry combinations.

In a further embodiment of the method, the method includes the surface mesh received from a three-dimensional (3D) body scanner.

In another embodiment of the method, the method includes the 3D body scanner generating the surface mesh.

In a further embodiment of the method, the method of processing the subregion geometries includes a regional body composition estimator multiplying subregion geometry combinations.

In another embodiment of the method, the method of summing the regional composition estimates produces the individual body composition.

In a further embodiment of the method, the method includes a regional body composition estimator retrieving the weights used to process the subregion geometries from a memory.

In another embodiment of the method, the method for the individual body composition includes an estimate of at least one of fat, lean, and bone mineral content as a portion of total body mass.

While implementations of the disclosure are susceptible to embodiment in many different forms, there is shown in the drawings and will herein be described in detail specific embodiments, with the understanding that the present disclosure is to be considered as an example of the principles of the disclosure and not intended to limit the disclosure to the specific embodiments shown and described. In the description above, like reference numerals may be used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” “includes,” “including,” “has,” “having,” or any other variations thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C.” An exception to this definition will occur only when a combination of elements, functions, steps or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

In the following description, it is understood that terms such as “first,” “second,” “top,” “bottom,” “up,” “down,” “above,” “below,” and the like, are words of convenience and are not to be construed as limiting terms. Also, the terms apparatus, device, system, etc. may be used interchangeably in this text.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A system, comprising:
a three-dimensional (3D) body scanner; and
a regional body composition estimator, coupled to the 3D body scanner, having a processor configured to:
generate one or more anatomical subregion geometries based, at least in part, on a surface mesh received from the 3D body scanner,
produce regional composition estimates by processing the subregion geometries by weights, and
produce an individual body composition from the regional composition estimates.

2. The system of claim 1, where the individual body composition includes an estimate of at least one of fat, lean, and bone mineral content as a portion of total body mass.

3. The system of claim 1, where processing the subregion geometries includes multiplying subregion geometry combinations.

4. The system of claim 1, further comprising a memory that stores the weights, where the regional body composition estimator processes the subregion geometries by weights received from the memory.

5. The system of claim 1, where the individual body composition is produced by summing the regional composition estimates.

6. A regional body composition estimator apparatus, comprising:
regional body composition estimator having:
a memory; and
a processor, coupled to the memory, configured to:
receive one or more anatomical subregion geometries and associated regional body compartment mass values from the memory,
fit a predictive model to the regional body compartment mass values using combinations of subregion groupings as inputs, and
determine the combination of subregion groupings having a best fit.

7. The regional body composition estimator apparatus of claim 6, the processor further configured to store the combination of subregion groupings having the best fit in the memory.

8. The regional body composition estimator apparatus of claim 6, where the memory and the processor of the regional body composition estimator apparatus reside within a body scanner that generates the one or more anatomical subregion geometries and associated regional body compartment mass values.

9. The regional body composition estimator apparatus of claim 6, where at least one or more of the memory and the processor of the regional body composition estimator apparatus reside within a computing device or where at least one or more of the memory and the processor reside within a distributed computing environment.

10. A method, comprising:
receiving one or more anatomical subregion geometries and associated regional body compartment mass values;
iteratively fitting a predictive model to the regional body compartment mass values using combinations of subregion groupings as inputs; and
determining the combination of subregion groupings having a best fit.

11. The method of claim 10, further comprising:
recording the combination of subregion groupings having the best fit in storage.

12. The method of claim 10, further comprising generating the one or more anatomical subregion geometries based, at least in part, on a surface mesh received from the 3D body scanner.

13. A method, comprising:
generating one or more anatomical subregion geometries based, at least in part, on a surface mesh;
producing regional composition estimates by processing the subregion geometries by weights; and
producing an individual body composition from the regional composition estimates.

14. The method of claim 13, where said processing the subregion geometries includes multiplying subregion geometry combinations.

15. The method of claim 13, the surface mesh received from a three-dimensional (3D) body scanner.

16. The method of claim 15, the 3D body scanner generating the surface mesh.

17. The method of claim 13, where processing the subregion geometries includes a regional body composition estimator multiplying subregion geometry combinations.

18. The method of claim 13, where summing the regional composition estimates produces the individual body composition.

19. The method of claim 13, further comprising a regional body composition estimator retrieving the weights used to process the subregion geometries from a memory.

20. The method of claim 13, where the individual body composition includes an estimate of at least one of fat, lean, and bone mineral content as a portion of total body mass.
